(19) United States
(12) Patent Application Publication
(10) Pub. No.: US 2016/0007922 A1
(43) Pub. Date: Jan. 14, 2016

(54) A METHOD AND SYSTEM FOR ESTIMATION OF BLOOD ANYLATES

(71) Applicants: Abhishek Sen, (US); Aman Midha, (US)

(72) Inventors: Abhishek Sen, Nashik, Maharashtra (IN); Aman Midha, Ludhiana, Punjab (IN)

(21) Appl. No.: 14/771,613

(22) PCT Filed: Mar. 3, 2014
(86) PCT No.: PCT/IN2014/000138

§ 371 Date: Aug. 31, 2015

(30) Foreign Application Priority Data
Mar. 1, 2013 (IN) 3554/MUM/2012

(51) Int. Cl. A61B 5/00; A61B 5/107; A61B 5/0205; A61B 5/1455; A61B 5/145

(52) U.S. Cl. CPC A61B 5/6826; A61B 5/1455; A61B 5/14546; A61B 5/02055; A61B 5/7475; A61B 5/742; A61B 5/7278; A61B 5/1079; A61B 2560/0475; A61B 2562/0238; A61B 2562/164

(57) ABSTRACT

The present invention relates to a method and system for estimation of blood anylates, more particularly the invention relates to mitigation strategies for the confounding factors in the non-invasive estimation of blood anylates. The present invention is hypothesized to measured with an accuracy reasonable for an anemia screening tool through measurement of the following parameters Finger Size, Transmitted infrared light through the finger, Finger tip temperature.

### BACKGROUND

1. Technical Field

The present invention relates to a method and system for estimation of blood anylates, more particularly the invention relates to mitigation strategies for the confounding factors in the non-invasive estimation of blood anylates.

2. Background of the Invention

In the real world, however, patients do complain about the constant poking and prodding that their physicians must subject them to in order to diagnose or monitor their conditions. Hence, many of the diagnostic manufactures are working to device products that are more patient-friendly.

For realizing new diagnostics technology suitable for day-to-day health management and disease prevention, the research and development in the field of biomedical engineering for non-invasive measurement of body constituents.

Progress in the development of non-invasive diagnostic technologies is being made along several fronts and for variety of conditions.

However, confounding is a major concern in causal studies because it results in biased estimation of exposure effects. In the extreme, this can mean that a causal effect is suggested where none exists, or that a true effect is hidden.

Generally, confounding occurs when there are differences between the exposed and unexposed groups in respect of independent risk factors for the disease of interest.

Any or all of the present systems and method thereof for non-invasive diagnostics cannot be used for accurate estimation of blood anylates due to many and all of the above mentioned constraints.

For the reasons stated above, which will become apparent to those skilled in the art upon reading and understanding the specification, there is a need in the art for a system and method for estimation of blood anylates with mitigation strategies for the confounding factors in the non-invasive estimation of blood anylates.

### BRIEF DESCRIPTION OF THE DRAWINGS

Reference will be made to embodiments of the invention, examples of which may be illustrated in the accompanying figures. These fingers are intended to be illustrative, not limiting. Although the invention is generally described in the context of these embodiments, it should be understood that it is not intended to limit the scope of the invention to these particular embodiments.

FIG. 1 illustrates view of a finger probe

FIG. 2 illustrates a tunnel built around the optical proximity sensor

FIG. 3 illustrates a plot of Finger size against the output from the silicon photodiode (ADC values)

Experimental data is collected from 150 volunteers in a general outpatient sample population. Sampling method is stratified sampling based on gender and age. For each volunteer, the following measurements are done:
a) Reference haemoglobin (Invasive collection via venepuncture and quantification using the cyanmeth, Drabkin's method). Units—g/dl
b) Finger size: Finger circumference at the middle of the distal phalynx with the help of a measuring tape. Units—mm
c) Skin colour: Quantification of skin colour with the help of a skin colour gradation chart which is used visually, where 1 represents the lightest skin colour and 10 represents the darkest skin colour.
d) Finger temperature: Fingertip temperature measured with the help of a NTC thermistor (0.1% tolerance). Units: degree farenheit
   Output of the silicon photodiode+940 nm LED in a transmittance pattern with a fixed current throughout the LED, ensuring constant illumination (LED and photodiode on opposite sides of the fingertip). Units: 0-4095 measured with a 12 bit ADC

FIG. 4 illustrates a plot of Total Hb in g/l against the output from the silicon photodiode (ADC values)

FIG. 5 illustrates a plot of Hb in g/l against Finger size (circumference) in mm.

FIG. 6 illustrates a plot of Skin colour (measured in grades of skin colour) against the output from the silicon photodiode (ADC values)

FIG. 7 illustrates a plot of Skin colour (measured in grades of skin colour) against size (circumference) in mm

FIG. 8 illustrates Absorption spectrum of melanin

FIG. 9 illustrates a plot of Finger circumference in mm against ADC values. Lowest line represents Reference Hb of 14 g/dl and the top most line represents Reference Hb of 7 g/dl. Each line in the middle is at a resolution of 1 g/dl. Eg. Y axis of 2000 for finger size 40 represents Hb of 14 g/dl. Y axis of 2000 for finger size 45 represents Hb of 12 g/dl

### SUMMARY THE INVENTION

The present invention relates to a method and system for estimation of blood anylates, more particularly the invention relates to mitigation strategies for the confounding factors in the non-invasive estimation of blood anylates. The system and the method thereof is configured to receive the signal inputs provided by the ADC, process the input signal received from ADC i.e. equivalent to the phototransistor output signal to determine the finger size. The finger size is determined through the relative referencing of the reference LUT's stored at the memory and error correction through representative correction set based on the finger tip temperature. The processor is configured to receive the signal inputs provided by the ADC, process the input signal received from ADC i.e. equivalent to the silicon photodiode output signal to determine the total haemoglobin by plotting the finger size against the ADC signal corresponding to the output of the silicon photodiode to determine which of the reference line the plot coincides and thus determining the value of the total haemoglobin based on the reference table stored as the LUTs at memory, a plot based on which is shown in the FIG. 9. The processor is configure to activate the output device such as display device, display device preferably a LCD display to display the resultant value of the haemoglobin.

### DETAILED DESCRIPTION OF THE INVENTION

The embodiments herein provide a method and system for estimation of blood anylates with mitigation strategies for the confounding factors in the non-invasive estimation of blood anylates. Further the embodiments may be easily implemented in various non-invasive diagnostics systems. The method of the invention may also be implemented as application performed by a stand alone or embedded system.

The invention described herein is explained using specific exemplary details for better understanding. However, the invention disclosed can be worked on by a person skilled in the art without the use of these specific details.

References in the specification to “one embodiment” or “an embodiment” means that a particular feature, structure, characteristic, or function described in connection with the embodiment is included in at least one embodiment of the invention. The appearances of the phrase “in-one embodiment” in various places in the specification are not necessarily all referring to the same embodiment.

Hereinafter, the preferred embodiments of the present invention will be described in detail. For clear description of the present invention, known constructions and functions will be omitted.

Parts of the description may be presented in terms of operations performed by a computer system, using terms such as data, state, link, fault, packet, and the like, consistent with the manner commonly employed by those skilled in the art to convey the substance of their work to others skilled in the art. As is well understood by those skilled in the art, these quantities take the form of data stored/transferred in the form of electrical, magnetic, or optical signals capable of being stored, transferred, combined, and otherwise manipulated through mechanical and electrical components of the computer system; and the term computer system includes general purpose as well as special purpose data processing machines, switches, and the like, that are standalone, adjunct or embedded.

According to an embodiment of the present invention, the method and system for non-invasive diagnostics is a system and method for estimation of blood anylates with mitigation strategies for the confounding factors in the non-invasive estimation of blood anylates.

It is painful to the patient while creating biohazards and the need for associated disposal procedures. A device that can quickly and accurately measure haemoglobin has many healthcare applications, such as in physical examinations, emergency departments, primary health care providers, medical specialists, in situ measurement of bleeding during surgery for determining transfusion triggers, and in home healthcare for the chronically ill and aging population. As per described herewith the present invention is a non-invasive haemoglobin measurement technology meant for measuring the haemoglobin concentration in the blood without drawing blood.

Haemoglobin is one of the most strongly absorbing chromophore in the near infra red region. There are four major forms of haemoglobin, Red Hb, Oxy Hb, Met Hb, and CO Hb. Out of these Oxy and Red Hb are the dominant forms in majority of conditions with the exceptions of methemoglobinemia and smokers/burn patients respectively.

As per one of the embodiment of the present invention, the fingers of the adult patients is subjected to the light of near infra red region and data pertaining to the finger size, colour skin, finger temperature, SpO2 and perfusion index may be collected. Probe Experimental data is collected from 150 volunteers in a general outpatient sample population. Sampling method is stratified sampling based on gender and age. For each volunteer, the following measurements are done:
Reference haemoglobin (Invasive collection via venepuncture and quantification using the cyanmeth, Drabkin's method). Units—g/dl
Finger size: Finger circumference at the middle of the distal phalynx with the help of a measuring tape. Units—mm
Skin colour: Quantification of skin colour with the help of a skin colour gradation chart which is used visually, where 1 represents the lightest skin colour and 10 represents the darkest skin colour.
Finger temperature: Fingertip temperature measured with the help of a NTC thermistor (0.1% tolerance). Units: degree farenheit
   Output of the silicon photodiode+940 nm LED in a transmittance pattern with a fixed current throughout the LED, ensuring constant illumination (LED and photodiode on opposite sides of the fingertip). Units: 0-4095 measured with a 12 bit ADC

The other embodiment of the present invention have finger probe as the integrated component of the system.

In one of the embodiment of the present invention, the system may have finger probe wherein the finger probe is constructed such that the finger can be inserted.

In one of the embodiment of the present invention, the system have finger probe wherein the finger probe is constructed such that the finger is inserted in a silicon rubber sleeve which is flexible and may expands or contract to accommodate different finger sizes.

As per one of the embodiment of the present invention, the flexible silicon rubber part of the sleeve is attached to a rigid framework which may encompass the rubber sleeve.

As per one of the embodiment of the present invention, the flexible silicon rubber part of the sleeve is attached to a rigid framework which encompass the rubber sleeve wherein the framework house a reflective optical proximity sensor on the top side and consisting of a narrow viewing angle—NIR LED and a phototransistor. The LED shine light on the top of a flat portion of the rubber sleeve and the reflected light be detected by the phototransistor. The amount of light reflected to phototransistor depend on factors such as the distance between the flat surface of the silicon rubber sleeve and the sensor, the reflectivity of the flat surface of the silicon rubber sleeve.

In one of the embodiment of the present invention the surface of the rubber sleeve have constant reflectivity.

In one another embodiment of the present invention the reflectivity of the surface of the rubber sleeve is constant thus ensuing the output of the phototransistor to be directly dependent on the distance between the flat top surface of the rubber sleeve and the optical sensor.

As per one of the embodiment of the present invention, the finger size is inversely related to the distance between the top of the rubber, sleeve and the optical proximity sensor housed in the framework since the rubber sleeve is flexible.

To prevent the effect of the ambient light on the optical proximity sensor, in one of the embodiment of the present invention, a tunnel is built around the optical proximity sensor.

As per one of the embodiment of the present invention, the flexible silicon rubber part of the sleeve is attached to a rigid framework which encompass the rubber sleeve wherein the rubber sleeve houses components such as but not limited to LEDs, silicon photodiode, thermistor. The LEDs may be used in a “transmittance” pattern where the LEDs and photodiode are placed on two different, parallel planes. The LEDs may be controlled in a way such that they emit light with three different intensities. The thermistor measures the fingertip temperature at the pulp of the finger.

There may be few confounding factors experienced during the measurement which may affect the transmission of infra red light through the finger. The confounding factors may be hypothesized such as but not limited to Total Hb concentration, Finger Size, Perfusion status in extremities, Colour of Skin, SpO2 status etc.

As per one of the embodiment of the present invention, the output of the phototransistor from the optical proximity sensor is captured using a ADC and converted to a number which is representative of the circumference of the finger inside the silicon rubber sleeve. Further, the output from the silicon photodiode is representative of the transmitted light through the finger. The output from the silicon photodiode is highly correlated to the finger size with a correlation coefficient of 0.6. The output from the silicon photodiode is highly correlated to total haemoglobin with a correlation coefficient of 0.48.

There is a possibility that the correlation seen between the output from the silicon photodiode and Total Hb be attributed to the multicollinearity existing between Total Hb, Finger size and the output from the silicon photodiode. To rule out this possibility FIG. 5 shows the relatively weak correlation between Finger size and Total Hb. It means that the change in the output from the silicon photodiode with respect to total Hb is not merely because of an existing relationship between the output from the silicon photodiode and size. A low correlation was seen between the output from the silicon photodiode and skin colour (FIG. 6), but it can be attributed to multicollinearity between the output from the silicon photodiode, Finger size and skin colour (FIG. 7). The two graphs (FIG. 6 and FIG. 7) confirm the multicollinearity between finger size, skin colour and the output from the silicon photodiode. Thus, it is inferred that skin colour is not a major absorber or contributor to the transmittance of infra red light through the finger. This is also understood from the absorption spectrum of melanin (FIG. 8) wherein it is evident that absorption spectrum of melanin becomes relatively insignificant above 700 nm.

The amount of blood in extremities depends on many factors, some of them being: response of the sympathetic, nervous system to ambient temperature, stress, baseline cathecholamine activity. The temperature gradient (Difference between body temperature and fingertip temperature) of the extremities can be used as a predictor for blood flow in finger.

gradient=0.2-5.7.log(flow)

As per system and method thereof of one of the embodiment of the present invention, the output from the silicon photodiode also depends on the blood flow through the finger, which can be predicted by the finger tip temperature.

As per one of the embodiment of the present invention though no significant relation is seen with SpO2 fluctuations, it may be necessary to note that the sample size had no abnormal SpO2 readings.

As per the embodiment of the present invention, system and method thereof of the present invention is hypothesized to measured with an accuracy reasonable for an anemia screening tool through measurement of the following parameters:

1. Finger Size

2. Transmitted infrared light through the finger

3. Finger tip temperature.

As per system and method thereof of one of the embodiment of the present invention, the effects of finger size in the measurement of the output from the silicon photodiode is quantified and removed through setting up relationship between multiple finger sizes and total haemoglobin against the output from the silicon photodiode Thus generating the reference table from the experimental data that is collected in a general outpatient sample population wherein reference haemoglobin is determined through Invasive collection via venepuncture and quantification using the cyanmeth, Drabkin's method. Units—g/dl and finger size is measured by measuring finger circumference at the middle of the distal phalynx with the help of a measuring tape. Units—mm. The relationship is shown as a plot in the FIG. 9 wherein the Lowest line represents Reference Hb of 14 g/dl and the top most line represents Reference Hb of 7 g/dl. Each line in the middle is at a resolution of 1 g/dl. Eg. Y axis of 2000 for finger size 40 represents Hb of 14 g/dl. Y axis of 2000 for finger size 45 represents Hb of 12 g/dl

As per system and method thereof of one of the embodiment of the present invention, Fingertip temperature is used to estimate blood flow and correct errors.

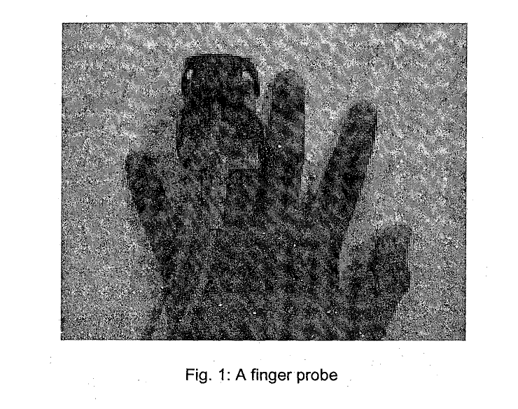

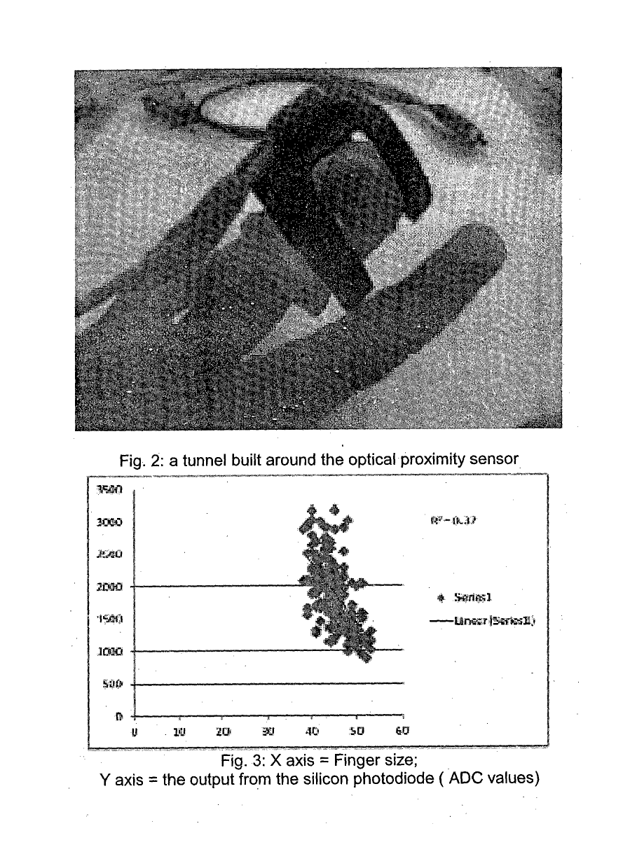

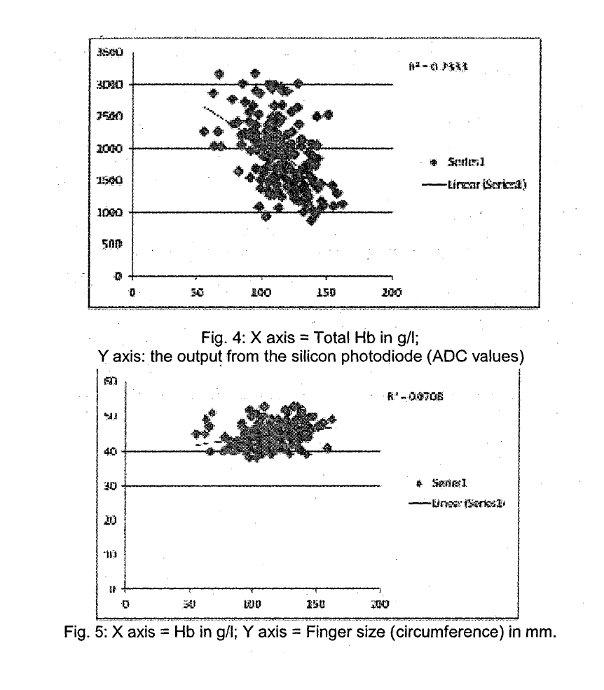

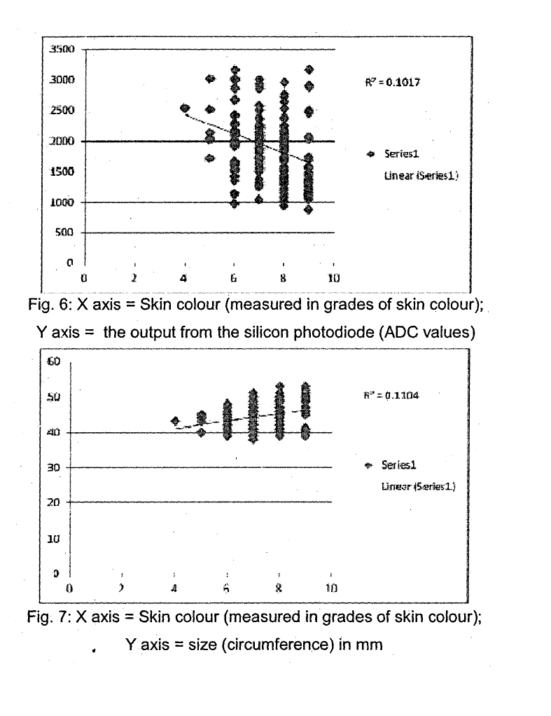

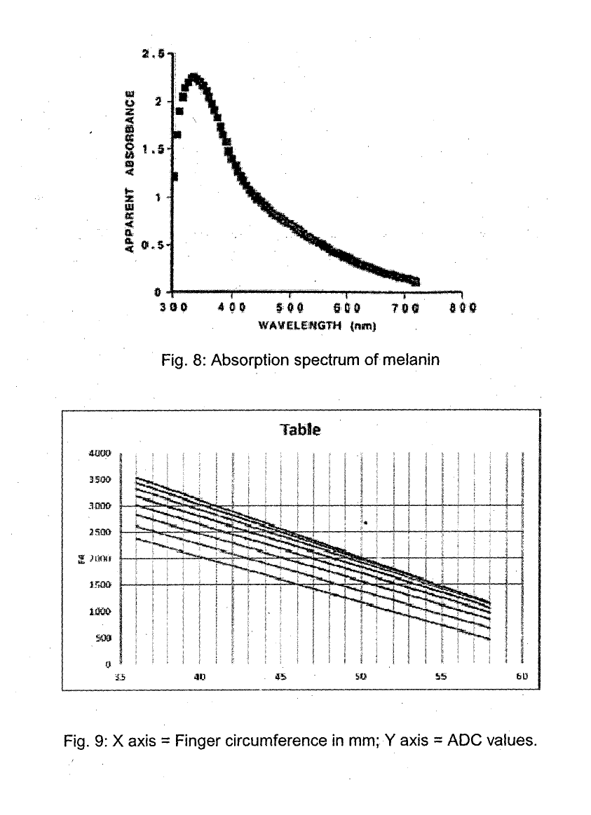

The system and the method thereof as disclosed in one of the embodiment of the present invention has a sensitivity of 79% and specificity of 76% to predict anemia in a non-invasive manner. As per one of the embodiment of the present invention, the system is comprising of a processor, at least one input device such as keyboard having at least one key for setting input, an output device such as display device, preferably LCD display, at least one. ADC, memory and a finger probe connected to the processor through probe wire to carry signals to the processor. The finger probe of the system comprise of flexible silicon rubber sleeve, a rigid ABS framework, a reflective optical proximity sensor (RPR-220) on the top side of the ABS frame work, consisting of a narrow viewing angle NIR LED and a phototransistor, inside of the rubber sleeve comprise at least Two LEDs of 660 and 940 nm, at least one silicon photodiode and at least one thermistor (NTC). The finger probe is constructed as per FIG. 1) where the finger is to be inserted in a silicon rubber sleeve which is flexible and expands to accommodate different finger sizes. The flexible silicon rubber part of the sleeve attaches to a rigid, ABS “framework” which encompasses the rubber sleeve. C) The ABS framework houses a reflective optical proximity sensor (RPR-220) on the top side, consisting of a narrow viewing angle NIR LED and a phototransistor. The NIR LED shines light on the top of a flat portion of the rubber sleeve and the reflected light is detected by the phototransistor. The amount of light reflected to the phototransistor depends on two factors, the distance between the flat surface of the silicon rubber sleeve and the sensor and the reflectivity of the flat surface of the silicon rubber sleeve. As the reflectivity of the surface of the rubber sleeve is remain constant, the output of the phototransistor directly depends on the distance between the flat top surface of the rubber sleeve and the optical sensor. Since, the rubber sleeve is flexible the finger size is inversely related to the distance between the top of the rubber sleeve and the optical proximity sensor housed in the ABS framework. To prevent effects of ambient light on the optical proximity sensor, a tunnel is built around the optical proximity sensor. Inside the rubber sleeve, are the following components, at least Two LEDs of 660 and 940 nm, at least one silicon photodiode and at least one thermistor (NTC). The two LEDs are used in a “transmittance” pattern where the LEDs and photodiode are on two different, parallel planes. The LEDs are controlled in a way such that they emit light with three different intensities. The NTC thermistor measures the fingertip temperature at the pulp of the finger. The ADC is configured to receive the analog output from the photodiode and silicon transistor and thermistor and to provide equivalent digital signal to the processor. The processor is configured to receive the signal inputs provided by the ADC, process the input signal received from ADC i.e. equivalent to the thermistor output signal. The processor is configured to receive the signal inputs provided by the ADC, process the input signal received from ADC i.e. equivalent to the phototransistor output signal to determine the finger size. The finger size is determined through the relative referencing of the reference LUT's stored at the memory and error correction through representative correction set based on the finger tip temperature. The processor is configured to to receive the signal inputs provided by the ADC, process the input signal received from ADC i.e. equivalent to the silicon photodiode output signal to determine the total haemoglobin by plotting the finger size against the ADC signal corresponding to the output of the silicon photodiode to determine which of the reference line the plot coincides and thus determining the value of the total haemoglobin based on the reference table stored as the LUTs at memory, a plot based on which is shown in the FIG. 9. The processor is configure to activate the output device such as display device, display device preferably a LCD display to display the resultant value of the haemoglobin.

As per one of the embodiment of the present invention the method is comprising steps of setting a finger in to the finger probe, on receiving initialize input from the key board initialize the probe to set on the a reflective optical proximity sensor placed on the top side of the ABS frame work, consisting of a narrow viewing angle NIR LED and a phototransistor and to set on, upon receiving initialize input from the key board initialize the comprise at least Two LEDs of 660 and 940 nm, at least one silicon photodiode and at least one thermistor (NTC) placed inside the rubber sleeve, collect the output signal generated by the phototransistor through ADC, collect the output signal generated by the silicon photodiode through ADC, collect the output signal generated by the thermistor through ADC, transmit the digital signal equivalent to the output received from photodiode, thermistor and silicon phototransistor to the processor, receive the signal inputs provided by the ADC at the processor, process the input signal received from ADC i.e. equivalent to the phototransistor output signal to determine the finger size, process the input signal received from ADC i.e. equivalent to the silicon photodiode output signal to determine the total haemoglobin by plotting the finger size against the ADC signal corresponding to the output of the silicon photodiode to determine which of the reference line the plot coincides and thus determining the value of the total haemoglobin based on the reference table stored as the LUTs at memory, activate the output device such as display device, display the resultant value of the haemoglobin.

As per one of the embodiment of the present invention the finger size is determined through the relative referencing of the reference LUT's stored at the memory and error correction through representative correction set based on the finger tip temperature.

As per one of the embodiment of the present invention the reference table is generated from the experimental data that is collected in a general outpatient sample population wherein reference haemoglobin is determined through Invasive collection via venepuncture and quantification using the cyanmeth, Drabkin's method. Units—g/dl and finger size is measured by measuring finger circumference at the middle of the distal phalynx with the help of a measuring tape. Units—mm.

As per one of the embodiment of the present invention the reference table relationship is plotted wherein the Lowest line represents Reference Hb of 14 g/dl and the top most line represents Reference Hb of 7 g/dl. Each line in the middle is at a resolution of 1 g/dl. Eg. Y axis of 2000 for finger size 40 represents Hb of 14 g/dl. Y axis of 2000 for finger size 45 represents Hb of 12 g/dl

The present invention as described in one of the embodiment can be advantageously pushed to any connected platform using the network/Cloud services.

1. A system for non-invasive estimation of blood anylates, the system comprise:
a finger probe, the finger probe comprise of a flexible silicon rubber sleeve, a rigid ABS framework, at least one reflective optical proximity sensor (RPR-220) on the top side of the ABS frame work, consisting of at least one narrow viewing angle NIR LED and at least one phototransistor, inside of the rubber sleeve comprise at least Two LEDs of 660 and 940 nm, at least one silicon photodiode and at least one thermistor (NTC) and at least one probe wire to carry signals;
at least one input device such as keyboard having at least one key for setting input,
at least one output device such as display device, preferably LCD display;
at least one ADC to receive the analog input from the at least one phototransistor, the at least one thermistor and the at least one silicon photodiode and convert it into equivalent digital signal and provide as output;
a memory;
a processor, the processor configured to receive the signal inputs provided by the ADC, process the input signal received from ADC i.e. equivalent to the thermistor output signal, process the input signal received from ADC i.e. equivalent to the phototransistor output signal to determine the finger size, perform error correction through representative correction set based on the finger tip temperature, process the input signal received from ADC i.e. equivalent to the silicon photodiode output signal to determine the total haemoglobin by plotting the finger size against the ADC signal corresponding to the output of the silicon photodiode to determine which of the reference line the plot coincides and thus determining the value of the total haemoglobin based on the reference table stored as the LUTs at memory, activate the output device such as display device, and display device preferably a LCD display to display the resultant value of the haemoglobin.

2. The system as claimed in claim 1 wherein the finger probe is constructed where the finger is to be inserted in a silicon rubber sleeve which is flexible and expands to accommodate different finger sizes.

3. The system as claimed in claim 1 wherein the flexible silicon rubber part of the sleeve attaches to a rigid, ABS “framework” which encompasses the rubber sleeve.

4. The system as claimed in claim 1 wherein the ABS framework houses a reflective optical proximity sensor (RPR-220) on the top side, consisting of a narrow viewing angle NIR LED and a phototransistor wherein the NIR LED shines light on the top of a flat portion of the rubber sleeve and the reflected light is detected by the phototransistor.

5. The system as claimed in claim 1 wherein the amount of light reflected to the phototransistor depends on two factors, the distance between the flat surface of the silicon rubber sleeve and the sensor and the reflectivity of the flat surface of the silicon rubber sleeve.

6. The system as claimed in claim 1 wherein the reflectivity of the surface of the rubber sleeve is remain constant,

7. The system as claimed in claim 1 wherein the output of the phototransistor directly depends on the distance between the flat top surface of the rubber sleeve and the optical sensor since, the rubber sleeve is flexible

8. The system as claimed in claim 1 wherein the finger size is inversely related to the distance between the top of the rubber sleeve and the optical proximity sensor housed in the ABS framework.

9. The system as claimed in claim 1 wherein to prevent effects of ambient light on the optical proximity sensor, a tunnel is built around the optical proximity sensor.

10. The system as claimed in claim 1 wherein the two LEDs Inside the rubber sleeve, are used in a “transmittance” pattern where the LEDs and photodiode are on two different, parallel planes.

11. The system as claimed in claim 1 wherein the LEDs are controlled in a way such that they emit light with three different intensities.

12. The system as claimed in claim 1 wherein the NTC thermistor measures the fingertip temperature at the pulp of the finger.

13. The system as claimed in claim 1 wherein The finger size is determined through the relative referencing of the reference LUT's stored at the memory

14. A method for non-invasive estimation of blood anylates, the method comprising steps of:
setting a finger in to a finger probe;
on receiving initialize input from the key board initialize the probe to set on at least one reflective optical proximity sensor placed on the top side of a ABS frame work consisting of at least one narrow viewing angle NIR LED and at least one phototransistor;
on receiving initialize input from the key board initialize the probe to set on at least Two LEDs of 660 and 940 nm, at least one silicon photodiode and at least one thermistor (NTC) placed inside the rubber sleeve;
collect the output signal generated by the phototransistor through ADC;
collect the output signal generated by the silicon photodiode through ADC;
collect the output signal generated by the thermistor through ADC;
transmit the digital signal equivalent to the output received from photodiode; thermistor and silicon phototransistor to the processor;
receive the signal inputs provided by the ADC at the processor;
process the input signal received from ADC i.e. equivalent to the phototransistor output signal to determine the finger size;
process the input signal received from ADC i.e. equivalent to the silicon photodiode output signal to determine the total haemoglobin by plotting the finger size against the ADC signal corresponding to the output of the silicon photodiode to determine which of the reference line the plot coincides and thus determining the value of the total haemoglobin based on the reference table stored as the LUTs at memory, activate the output device such as display device;
display the resultant value of the haemoglobin.

15. The method as claimed in claim 14 wherein the finger size is determined through the relative referencing of the reference LUT's stored at the memory and error correction through representative correction set based on the finger tip temperature.
The reference table is generated from the experimental data that is collected in a general outpatient sample population wherein reference haemoglobin is determined through Invasive collection via venepuncture and quantification using the cyanmeth, Drabkin's method. Units—g/dl and finger size is measured by measuring finger circumference at the middle of the distal phalynx with the help of a measuring tape. Units—mm.

16. The method as claimed in claim 14 wherein the reference table relationship is plotted wherein the Lowest line represents Reference Hb of 14 g/dl and the top most line represents Reference Hb of 7 g/dl. Each line in the middle is at a resolution of 1 g/dl. Eg. Y axis of 2000 for finger size 40 represents Hb of 14 g/dl. Y axis of 2000 for finger size 45 represents Hb of 12 g/dl

17. The method as claimed in claim 14 wherein the amount of light reflected to the phototransistor depends on two factors, the distance between the flat surface of the silicon rubber sleeve and the sensor and the reflectivity of the flat surface of the silicon rubber sleeve.

18. The method as claimed in claim 14 wherein the reflectivity of the surface of the rubber sleeve is remain constant,

19. The method as claimed in claim 14 wherein the output of the phototransistor directly depends on the distance between the flat top surface of the rubber sleeve and the optical sensor since, the rubber sleeve is flexible

20. The method as claimed in claim 14 wherein the finger size is inversely related to the distance between the top of the rubber sleeve and the optical proximity sensor housed in the ABS framework.

21. The method as claimed in claim 14 wherein The LEDs are controlled in a way such that they emit light with three different intensities.

22. The method as claimed in claim 14 wherein the NTC thermistor measures the fingertip temperature at the pulp of the finger.

23. The method as claimed in claim 14 wherein the correlation coefficient is determined between finger size, ADCsignal value, Hb in g/l, skin color captured through experimental data collected from general outpatient sample population.

24. The method as claimed in claim 14 wherein based on correlation coefficient plots, it is hypothesized to measure with an accuracy reasonable for an anemia screening, only parameters that to be measured are finger size, transmitted infrared light through the finger and finger tip temperature and determine reference table that is to be stored as reference LUTs at memory,
